(19) United States
(12) Patent Application Publication
(10) Pub. No.: US 2018/0371061 A1
(43) Pub. Date: Dec. 27, 2018

(54) APPLICATION OF ZN7MT3 AND ITS DERIVATIVES IN THE PREVENTION AND TREATMENT OF ALZHEIMER'S DISEASE

(71) Applicant: LIAOCHENG CITY ORIENT BIOMEDICAL TECHNOLOGY CO., LTD, Liaocheng (CN)

(72) Inventors: Yingxuan TAN, Liaocheng (CN); Xiangshi TAN, Liaocheng (CN); Wei XU, Liaocheng (CN); Yueru ZHANG, Liaocheng (CN); Daofeng XIANG, Liaocheng (CN)

(21) Appl. No.: 16/103,914

(22) Filed: Aug. 14, 2018

Related U.S. Application Data
(63) Continuation of application No. PCT/CN2017/072805, filed on Feb. 3, 2017.

(30) Foreign Application Priority Data
Feb. 15, 2016 (CN) 201610085825.7

(51) Int. Cl. C07K 14/825; A61P 25/28; C07K 14/035; C12N 15/70

(52) U.S. Cl. CPC C07K 14/825; A61P 25/28; C07K 1/14; C12N 15/70; C07K 2319/24; C07K 14/035

(57) ABSTRACT

The present disclosure provides a use based on Zn₇MT3 or a derivative thereof. Zn₇MT3 or a derivative thereof is used for prevention or treatment of Alzheimer's disease or other neurodegenerative diseases, or for development, screening or preparation of a medicament suitable for Alzheimer's disease or other neurodegenerative diseases. Further provided are a method for preparing Zn₇MT3 and a method for preparing gH625-Zn₇MT3. Zn₇MT3 and the derivatives thereof of the present disclosure can be used for improving cognitive dysfunction of an AD brain, regulating the cellular morphology of hippocampus in the AD brain, inhibiting the deposition of the amyloid protein in the AD brain and inhibiting the apoptosis of nerve cells in the brain, and can effectively prevent the progression of senile dementia; and the method for preparing Zn₇MT3 is simple and efficient, and gH625-Zn₇MT3 can easily cross the blood-brain barrier.

### CROSS-REFERENCE TO RELATED APPLICATIONS

This application is a continuation of International Application No. PCT/CN2017/072805 with a filing date of Feb. 3, 2017, designating the United States, now pending, and further claims to Chinese Application No. 201610085825.7 with a filing date of Feb. 15, 2016. The content of the aforementioned applications, including any intervening amendments thereto, are incorporated herein by reference.

### TECHNICAL FIELD

The present disclosure relates to the technical field of biomedicine, and particularly to the use of Zn₇MT3 and its derivatives in resisting Alzheimer's disease and in the preparation of a medicament for resisting neurodegenerative diseases such as Alzheimer's disease.

### BACKGROUND

Alzheimer's Disease (AD), commonly known as senile dementia, was first reported in 1907 by a German scholar, Alosi Alzheimer. It is a primary degenerative disease of the central nervous system which often occurs in the elderly. The incidence of AD is closely related to age, the incidence of AD increases at an annual rate of 0.5% in people over 65 years of age, and the incidence of AD is higher in women than in men. At present, there are approximately 47.5 million AD patients in the world, whose clinical manifestations are memory loss at different levels, language difficulty, disorientation, cognitive decline, abnormal personality, behavior and emotional activity, and progressive mental retardation, ultimately leading to systemic failure and death from complicated infection. At present, AD has become the fourth cause of death following heart disease, tumors and strokes. The most typical pathological features of AD are: in cerebral cortex and hippocampus, there occur a large number of senile plaques (SP) and neurofibrillary tangles (NFTs), decreased number of neurons, granular-vacuolar degeneration and chronic neuroinflammation. The pathogenesis of AD is very complex, which may be the result of the interaction of many factors. Up to now, the exact pathogenesis thereof is still unknown, but the research evidence in the past 30 years suggests that amyloid-beta peptide (Abeta), amyloid precursor protein (APP), metallothionein (MT3), and the metal ions regulated thereby and the associated ROS homeostatic balance are closely related with the occurrence and development of AD.

The neuropathological mechanism of AD has been studied extensively at home and abroad. Since the mid-1970s, numerous pharmacological studies have focused on increasing acetylcholine levels in synaptic clefts, and a number of drugs of acetylcholinesterase inhibitors have been discovered. However, this treatment is only a palliative treatment for relieving the symptoms. Since the mid-1980s, many studies have focused on the formation, aggregation and clearance mechanisms of Abeta, or the neurotoxic mechanism of Abeta. Abeta-targeted drugs for the treatment of AD disease include inhibitors of Abeta precursor protein APP secretase, metal ion chelators resisting Abeta aggregation, Abeta antibodies, etc. However, in 2008, Lancet reported that Abeta vaccines could effectively clear Abeta plaques from the brain, but could not prevent the development of dementia symptoms. This report raised questions about the treatment research on Abeta plaques. These results suggest that Abeta plaques themselves are not the main source of neurotoxicity, and the possible mechanism is that ROS produced during the formation of Abeta plaques is toxic to nerve cells. By means of synchrotron radiation X-ray fluorescence analysis, it is found that there are high concentrations of metal ions in senile plaques, e.g., the contents of copper, iron and zinc ions are as high as 1.0, 0.4 and 1.0 mM, respectively. The abnormal high-concentration aggregations of these transition metal ions and the abnormal aggregations of the associated proteins (such as Abeta, tau proteins, etc.) in the brain are common features of various neurodegenerative diseases. Therefore, the field in which metal ions are highly related to neuromedicine draws great attention from scientists and neuroscientists. Extensive research reports have shown that homeostatic imbalance of some transition metal ions (Cu, Fe, Zn) in cranial nerves is one of the main causes of neuronal death in AD patients. The homeostasis regulation of these transition metal ions in the brain, especially Cu and Fe ions allowing for single electron redox, and the regulation of the related reactive oxygen species (ROS) level are likely to be effective strategies for the treatment of AD disease.

Metallothionein-3 (MT3), also known as neuronal growth inhibitory factor (GIF), is specifically expressed in the human brain. MT3 is composed of 68 amino acids, including 20 conserved cysteines. The two domains thereof contain two metal clusters, which can bind to a total of 7 divalent metal ions, i.e., M₃S₉ in the N-terminal beta-domain and M₄S₁₁ in the C-terminal Alpha-domain. The two domains are linked with three amino acids (KKS). In the brain, MT3 is largely distributed in neural astrocytes, but the expression quantity of MT3 is significantly decreased by about one-third in the brain of AD patients. Impairment of MT3 expression may be associated with the onset of AD symptoms or neurological function impairment. MT3 can also convert a NO signal to a zinc ion signal. By direct S-nitroso reaction between NO and cysteine binding to zinc in MT3 or by nitroso conversion reaction between NO and S-nitrosothiol, zinc ions are released from MT3. Therefore, MT3 supplementation is one of the effective strategies for the prevention and treatment of AD. However, MT3 is a polypeptide having a molecular weight of 7000, which can hardly cross the blood-brain barrier.

Zinc ions are messengers in the brain, and brain has the highest zinc content, most typically up to about 150 μM in grey matter. In the brain, zinc in the form of free ions is abundantly concentrated in many glutamatergic nerve terminals (10-15%). When zinc is released and enters the synaptic clefts, the ion concentration in the synaptic clefts may rise to the order of millimoles. Like copper ions, the released zinc ions interact with neuroreceptors such as NMDA in the synaptic clefts and also act on various neuronal ion channels and transporters to regulate neural signaling. A variety of zinc transport proteins (ZnTs), MTs, etc. bind to the zinc ions in the cytoplasm, thereby preventing free zinc ions from becoming toxic. Compared with copper ions, the content of zinc ions in plasma decreases gradually after birth, and the content of zinc ions in the plasma of the AD patients is further decreased as compared with that in healthy people of the same age. Although the total content of zinc ions has no connection with aging of the brain, it is known that certain regions containing high concentrations of zinc ions, e.g., the hippocampal region dominated by high glutamate, exhibit a decrease in zinc ion content as the age increases. It is reported in a number of current literatures that zinc supplementation of the brain may be one of the effective strategies to prevent and treat senile dementia.

As early as 1998, Lovell et al. confirmed the presence of numerous zinc ions, up to about 1 mM, in certain regions (e.g., amyloid plaques, commonly known as senile plaques) of an AD brain, while the content in normal nerve fibers of the same age was merely about 350 μM. In addition, the expression levels of Zn transport proteins, such as ZnT1, ZnT4 and ZnT6, were found to have been changed in AD patients. These findings cause people to pay attention to the potential role of Zn in the pathological process of AD.

Zinc ions are also closely related with the aggregation of amyloid-beta peptide Abeta, which can quickly cause Abeta to form a precipitate that cannot be degraded by protease. Under in vitro simulated physiological conditions, zinc ions bind to Abeta at a ratio of 1:1 to form a complex, while the structure of aggregate is considered to be more amorphous, e.g., with lower fiber content. However, this activity may not be neurotoxic, but is considered to be of neuroprotective effect, as in vitro cortical nerve cell culture experiments demonstrate that zinc ions can reduce Abeta-induced cytotoxicity. The exact mechanism of zinc ion protection against Abeta toxicity is still not known, but one possibility is that Zn competes with Ca or Fe metal ions in binding to Abeta, which causes it to change its conformation so that Cu/Fe ions cannot contact their metal binding sites, thereby preventing them from binding to Abeta, further preventing the generation of hydrogen peroxide and free radicals.

Metallothionein MT3 is the main source of zinc in neurons. Recent studies have shown that zinc ions binding to MT3 can undergo metal replacement with Abeta-Cu complex, thereby inhibiting oxidative damage caused by Abeta reducing Cu²⁺. The imbalance of zinc ions in AD brains may result from the inhibition to zinc ion output, and a peroxide 4-hydroxynoenal (4-hydroxy barbiturate) produced due to Abeta-Cu reduction activity is thought to have this effect. Consistent with this, mouse animal studies have shown that the systemic zinc ion loss causes the retention of zinc ions in the brain, and this effect results from the inhibition to the intracellular zinc ion output protein ZnT1.

In the synapses, MT3, Abeta and copper ions may form a dynamic balance. Under the action of ZnT3, Zn²⁺ and glutamic acid aggregate in the presynaptic vesicles at the same time. The concentration of Zn²⁺ in the synaptic clefts is as high as 0.3 mM. The NMDA-mediated activation causes the copper ions to be released after synapsis and transported to the synaptic clefts, and then the concentration of copper ions in the synaptic clefts reaches the order of millimoles. Copper and zinc can in turn inhibit the NMDA receptor response. After being enzyme digested by amyloid precursor protein (APP) and then released into the synaptic clefts, Abeta can react with copper in the clefts, and then cross-linked therewith to form soluble Abeta aggregates and even amyloid precipitates. MT3 is released from adjacent astrocytes and enters the synaptic clefts, which can alleviate the adverse reaction. Neurometallic ions, Abeta and MT3 form a dynamic balance in the synaptic clefts, and this balance can prevent Abeta from forming fibrous precipitates in the synaptic clefts. Proper neural synaptic activity may promote this balanced system, but excessive or abnormal neural activity may be detrimental to this system. To regulate cranial nerves [metal ions-Abeta-MT3] to form a beneficial balance may be an innovative approach to the treatment of senile dementia, which is of important significance for the regulation and treatment of the AD disease.

Maintaining the homeostatic balance of reactive oxygen species (ROS) in brain is a necessary condition for normal physiological functions of brain. Brain consumes a fifth of the human body's oxygen, and antioxidants and related enzymes are relatively low in concentration and are rich in unsaturated fatty acids, which are prone to oxidative damage. Under normal conditions, cells can resist oxidation attack by regulation of homeostatic balance, but with the increase of age, the ability of the cells to maintain homeostatic balance decreases, leading to free radical accumulation, mitochondrial dysfunction and neuronal damage. Oxidative stress occurs when the number of ROS goes beyond the ability of neuronal cells to cope, leading to mitochondrial dysfunction and neuronal cell damage. The lack of histones in mitochondria and the weakening of DNA repair function in mitochondria are both the causes of oxidative stress in mitochondria. Copper ions are considered as an important factor for the occurrence of oxidative stress in AD. In the AD disease, the increase of oxidative damage is not the final result, but has an effect of initiation. In the early stage of AD, neurons are actually still in a homeostatic balance despite the increase of oxidative damage. As the AD disease develops and the corresponding ROS level increases, Abeta-metal compounds and hyperphosphorylated tau proteins will not be effectively eliminated, leading to an uncontrollable increase in amyloid plaques and neurofibrillary tangles, which in turn leads to a further increase in reactive substances, and this deteriorating feedback mechanism ultimately results in loss of neuronal functions.

### SUMMARY

The inventor has studied the regulation function of Zn₇MT3 in cranial nerves through AD mouse models, and has found that Zn₇MT3 can improve the cognitive and memory abilities of AD model mice, inhibit apoptosis of nerve cells, inhibit amyloid protein deposition, aggregation, etc., improve the cognitive and memory abilities, and can effectively present the progression of senile dementia.

Based on the above findings, in order to supplement the metal homeostasis regulatory protein lacking in the brain with AD disease and increase the neurotrophic element zinc, the inventor prepared a fusion protein gH625-MT3 by recombination of a transmembrane peptide gH625 capable of passing through the cell membrane and the blood-brain barrier with MT3, and then obtained, by metal recombination fusion, gH625-Zn₇MT3 which can effectively pass through the blood-brain barrier. The inventor studied the homeostasis regulation function of gH625-Zn₇MT3 in cranial nerves through AD transgenic mouse models, and found that gH625-Zn₇MT3 can improve the cognitive and memory abilities of AD model mice, inhibit apoptosis of nerve cells, inhibit amyloid protein deposition, etc., and can effectively prevent the development of senile dementia.

Accordingly, an object of the present disclosure is to provide a use based on Zn₇MT3 and a derivative thereof and the solution is as follows: a use based on Zn₇MT3 or a derivative thereof. Zn₇MT3 or a derivative thereof is used for prevention or treatment of Alzheimer's disease or other neurodegenerative diseases, or for development, screening or preparation of a medicament suitable for Alzheimer's disease or other neurodegenerative diseases.

Preferably, Zn₇MT3 or a derivative thereof is used for improving cognitive dysfunction of an AD brain, regulating the cellular morphology of hippocampus in the AD brain, inhibiting the deposition of amyloid proteins in the AD brain or inhibiting the apoptosis of nerve cells in the brain.

Preferably, the dosage form of the medicament includes at least one of tablets, capsules, granules, suspensions, emulsions, solutions, syrups and injections.

Preferably, the derivative of Zn₇MT3 includes gH625-Zn₇MT3 , or other similar fusion proteins based on metallothionein MT3 or Zn₇MT3 fused with transmembrane small peptide tags.

Another object of the present disclosure is to provide a method for efficiently preparing Zn₇MT3, and the solution is as follows: a method for efficiently preparing Zn₇MT3, including the steps of:

S1, fusion-expressing MT3 with MBP and Smt3 tags; and

S2, subjecting the semi-finished product obtained in S1 to acid denaturation to remove impurity metals, then adding thereto excess zinc ions to renature MT3 protein, and subjecting the resultant product to separation and purification to remove the surplus zinc ions, thereby obtaining Zn₇MT3.

When MT3 is fusion-expressed by MBP and Smt3 tags, the soluble expression efficiency in Escherichia coli is 6 times as high as that in the case where the tags are not fused, and the purity of purification of the protein is 10% higher.

Preferably, MT3 is solubly expressed in Escherichia coli.

A further object of the present disclosure is to provide a brain metal homeostasis regulatory protein which can conveniently pass through the blood-brain barrier. The preparation solution thereof is as follows: a method for preparing gH625-Zn₇MT3, wherein gH625-Zn₇MT3 is made by metal recombination of gH625-MT3 formed by fusion of gH625 and MT3.

Preferably, gH625 includes a transmembrane sequence in a glycoprotein of herpes simplex virus, the transmembrane sequence contains 23 amino acid residues, MT3 includes metallothionein III, and metallothionein III contains 68 amino acid residues.

Preferably, a Smt3-MT3 gene expression plasmid containing a fusion tag is constructed by using a vector through the genetic engineering technology, the gene sequence of gH625 is inserted into the Smt3-MT3 gene to form a smt3-gH625-MT3 fusion protein gene, the smt3-gH625-MT3 recombinant fusion protein is solubly expressed in Escherichia coli, and is then subjected to separation and purification to obtain gH625-MT3 recombinant fusion protein, which is further caused to bind to zinc ions by chemical recombination, thereby obtaining gH625-Zn₇MT3.

Preferably, the vector is pET22b(+).

Zn₇MT3 and the derivatives thereof of the present disclosure can be used for improving cognitive dysfunction of an AD brain, regulating the cellular morphology of hippocampus in the AD brain, inhibiting the deposition of the amyloid protein in the AD brain, and inhibiting the apoptosis of nerve cells in the brain, and can effectively prevent the progression of senile dementia; and the method for preparing Zn₇MT3 is simple and efficient, and gH625-Zn₇MT3 can easily cross the blood-brain barrier.

### BRIEF DESCRIPTION OF THE DRAWINGS

FIG. 1: Regulatory effect of Zn₇MT3 on the cellular morphology in CA1 region of brain tissue hippocampus of AD mice;

FIG. 2: Zn₇MT3's inhibition on the deposition of Abeta proteins in the brain of the AD mice;

FIG. 3: Impact of Zn₇MT3 on the apoptosis of nerve cells in the brain of the AD mice;

FIG. 4: Regulatory effect of gH625-Zn₇MT3 on the cellular morphology in CA1 region of brain tissue hippocampus of the AD mice;

FIG. 5: gH625-Zn₇MT3's inhibition on the deposition of Abeta proteins in the brain of the AD mice; and

FIG. 6: Impact of gH625-Zn₇MT3 on the apoptosis of nerve cells in the brain of the AD mice.

In each of the figures, A is a control group (normal mice), B is an AD mice model group, and C is AD model mice administered with a medicament for six weeks.

### DETAILED DESCRIPTION

The present disclosure will be described in detail by way of examples. In the present disclosure, the embodiments described below are intended to illustrate the present disclosure better, rather than limit the scope of the present disclosure.

It should be noted that the mentioned human MT3 is a specific protein name, which, if not specified, is consistent with most published documents, the NCBI database and the European gene database.

Human MT3 protein amino acid sequence (1-68):

MDPETCPCPS GGSCTCADSC KCEGCKCTSC KKSCCSCCPA
ECEKCAKDCV CKGGEAAEAE AEKSSCCQ.

MT3 (Metallothionein 3), also referred to as neuronal growth inhibitory factor, contains 68 amino acid residues, with the GenBank name EAW82868.1.

GH625-MT3 fusion protein amino acid sequence (1-91):

HGLASTLTRW AHYNALIRAF GGGMDPETCP CPSGGSCTCA
DSCKCEGCKC TSCKKSCCSC CPAECEKCAK DCVCKGGEAA
EAEAEKSSCC Q.

### Embodiment 1: Preparation of Zn₇MT3

Unless otherwise specified, all biochemical reagents and kits were purchased from Sigama or Invitrogen.

(1) Plasmid Construction

Human metallothionein MT3 and fusion tag protein gene Smt3 were purchased from Guangzhou Funeng Gene Company, the gene vector plasmid MBPHT-mCherry-2 was purchased from Invitrogen, and the synthesis of the gene primers was completed by Shanghai Generay Biotech Co., Ltd. A PCR product was obtained by amplification using the designed primers P1, P2 (amplifying Smt3 gene) and P3, P4 (amplifying MT3 gene). The amplification product was verified by 1% agarose gel detection and the desired fragment was recovered with gel. Product 1 and product 2 were mixed at the same concentration ratio to serve as a template, and the Smt3-MT3 sequence was obtained by amplification using P1 and P4 primers, with its 5′ end carrying BamHI enzyme digestion site GGATCC and its 3′ end carrying HindIII enzyme digestion site AAGCTT. The designed amplification primers were as follows:

P1:
5′-CGCGGATCCATGGCTAGCATGTCGGACTC-3′
P2:
5′-GGCAGGTCTCAGGGTCCATACCACCAATCTGTTCTC-3′
P3:
5′-GAGAACAGATTGGTGGTATGGACCCTGAGACCTGCC-3′
P4:
5′-CGCAAGCTTTCACTGGCAGCAGCTGCA-3′

The vector MBPHT-mCherry-2 and the amplified Smt3-MT3 gene sequence were subjected to double enzyme digestion at 37° C. for 6 hours by using BamHI and HindIII endonucleases, respectively, enzyme digestion was verified to be successful by means of 1% agarose gel detection, and the digested fragments were recovered with gel. The digested large and small fragments were mixed at 1:3 and ligated at 16° C. for 8 hours with T4 ligase. The ligation product was transformed into Top10 clone competent cells, and positive clones were picked for PCR identification and sequencing to prove successful construction of the fusion protein MT3-Smt3 expression plasmid.

(2) Expression and Purification of MT3 Protein

The constructed plasmid was transformed into host bacterium BL21 (DE3). Single colonies were picked out and placed in 3 ml of LB culture medium, were cultured in a shaker at 37° C. for 8 hours, then inoculated into 50 ml of LB culture medium at a ratio of 1:100 and cultured in a 37° C. shaker for 6 hours, and further inoculated into 2YT culture medium at a ratio of 1:100 (the abovementioned culture mediums all contained 100 μg/ml ampicillin sodium). When the colonies were cultured in a shaker at 37° C. and at 200 rpm to have an OD value of 0.6-0.8, the IPTG inducer was added thereto until the final concentration reached 0.4 mM, and the resultant product was expressed overnight at 16° C.

The purification process of the fusion protein was as follows: the bacteria harvested through centrifugation were suspended (1 ml/g bacteria) by adding thereto Tris buffer solution (20 mM Tris-HCl, 500 mM NaCl, 10% glycerol, 5 mM β-mercaptoethanol, pH=7.5), a small amount of lysozyme, DNA enzyme and 1% PMSF were added thereto, the bacteria were dissolved by stirring and disrupted with an ultrasonic disintegrator, and then centrifuged with a high-speed centrifuge (for 30 min at 12000 rpm), and the supernatant was taken therefrom to flow through a well-balanced affinity Ni-NTA column so that the His-tagged fusion proteins were attached to the column. The impurity proteins were eluted with Tris buffer solution containing 20 mM imidazole until no color change was detected by Coomassie Brilliant Blue. The fusion proteins were eluted with Tris buffer solution containing 200 mM imidazole. The eluted fusion proteins were placed into a dialysis bag for dialysis, the dialysate was Tris buffer solution, 1 L was used each time, the dialysate was changed every 6 hours for three times in total, thereafter SUMO enzyme was added thereto at a ratio of 1% for enzyme digestion for 6 hours, the resultant product was flowed through the well-balanced Ni-NTA column for three times in total to remove tag proteins and SUMO enzyme, and then subjected to Superdex-G75 gel molecular sieve to further remove the impurity proteins, and the purified MT3 protein was detected, by 15% SDS-PAGE gel detection, to have a purity of 95% or more.

(3) Metal Recombination of Zn₇MT3

DTT was added to 2 ml of MT3 protein solution (5 mg/ml) until the final concentration reached 50 mM, and the mixture was reduced at 4° C. for 2 hours. 6M HCl solution was added thereto to regulate the pH to 1-2, then the mixture was acidified for 1 hour and then was passed through the Superdex G-25 column to remove impurity metal ions, and MT3 protein was eluted with HCl solution having the pH of 2.0. The effluent was detected by an ultraviolet spectrometer and then collected. Demetallized Apo-MT3 protein solution was loaded into anaerobic glove box after being degassed, DTT was added thereto until the final concentration reached 50 mM, ZnCl₂ having a concentration 20 times the protein concentration was added thereto, 2M Tris base was added thereto dropwise slowly to regulate the pH to 8-9, the product was left overnight at room temperature, and then dialyzed to remove surplus zinc ions, and Zn₇MT3 was obtained after protein concentration.

(4) Property Characterization of Zn₇MT3

The MT3 protein concentration calibration method: 2,2′-dithiodipyridine (2-PDS) colorimetry was adopted. The principle was that the sulfhydryl group in the protein was oxidized by using 2,2′-dithiodipyridine and the resultant product 2-thiopyridine had sharp absorption at 343 nm. 10 μl of protein solution was added to 500 μl of determination solution (2 mM 2,2′-dithiodipyridine, 1 mM EDTA, 0.2 M sodium acetate, pH 4.0). The mixture was mixed well and stood at room temperature for 5 minutes, and then the value of absorbance at 343 nm was measured with an ultraviolet spectrometer and the concentration of the sulfhydryl group (—SH) in the protein was calculated using the lamber-beers law. C(—SH)=A₃₄₃/(ε₃₄₃·L), where A₃₄₃ was the absorbance of the reaction product (2-thiopyridine) at 343 nm, ε₃₄₃ was the molar extinction coefficient (7.06×103 M⁻¹) of the reaction product at 343 nm, and L was the optical diameter length (cm). Since the metallothionein contains a plurality of sulfhydryl groups, the concentration of the protein could be obtained just by dividing the measured concentration of sulfhydryl groups by the number of sulfhydryl groups.

Metal content determination of Zn₇MT3: a small amount of concentrated nitric acid was added to a certain amount of recombinant protein for nitrification overnight at 65° C., the resultant product was tenfold diluted, and then the content of metal Zn was measured on an inductively coupled plasma optical emission spectrometer (ICP-OES). The results showed that per mole of recombinant MT3 protein contained 7±0.2 moles of Zn.

(5) Removal of Endotoxin from Zn₇MT3

Zn₇MT3 (with a molecular weight of about 7 KD) was first subjected to 10 KD ultrafiltration membrane to entrap endotoxin (LPS) in aggregation state, and then subjected to Polymyxin B affinity column to remove the residual endotoxin.

### Embodiment 2: AD Mouse Model and Zn₇MT3 Drug Therapy

APP/PS1 transgenic mice were purchased from Beijing Zhongke Zesheng Biotechnology Co., Ltd., 5 months old, weighing 24-26 g. Name of the test drug: Zn₇MT3; solvent: normal saline; and preparation method: preparing the drug into a solution at the desired concentration with normal saline solution just before use. Intraventricular administration of mice: the experimental animals were APP/PS1 transgenic mice. Sustained release administration was performed for 6 weeks by using an Alzet miniosmotic pump Model 2006. The administration site was the lateral ventricle, at the time of implanting a catheter, a stereotactic instrument was used for precise positioning (with the anterior fontanelle being the origin, 1.0 mm to the left or right, 0.4 mm to the back, and 0.3 mm deep), the dosage was 2 mg/kg/day, and 6 weeks later, the blood serum and brain tissues were harvested.

### Embodiment 3: Verification of Cognitive Ability of AD Mice Using Morris Water Maze

Installation: a round pool was used, having a diameter of 1 m, a height of 50 cm and water depth of 30 cm, and having a white bottom, and water temperature was maintained at 23±2° C. Four equally spaced points N, E, S and W were marked on the wall of the pool and served as the starting points of the test. The pool was divided into four quadrants, and a platform was placed in the center of the third quadrant (the platform was equidistant from the wall of the pool and the circle center). The platform was submersed in water at 1 cm depth, making the platform invisible. Lots of clues (triangles, squares, circles and rhombuses in different colors were placed in the quadrants) were arranged around the pool and the clues remained unchanged for use by the mice to position the platform. Place navigation test: the test lasted for 6 days, and training was performed for 4 times at fixed time periods every day. At the beginning of the training, the platform was placed in the first quadrant, and the mice were placed into the pool facing the wall of the pool from any of the four starting points on the wall of the pool. The time it took for the mice to find the platform and the swimming path of the mice were recorded by a free video recording system, and the four times of training referred to the four training sessions started by placing the mice in water from the four different starting points (different quadrants). After the mice found the platform or if the mice could not find the platform within 90 seconds (the latent period was set to 90 seconds), the experimenter directed the mice to the platform, the mice rested on the platform for 10 seconds, and then the next test was started.

Spatial probe test: the platform was removed 24 h after the place navigation test was completed. The mice were then placed into water from the third quadrant, the swimming paths of the mice within 180 s were recorded, the residence time of the mice in the target quadrant (the third quadrant) and the frequency at which the mice crossed the original position of the platform were recorded, and the spatial positioning ability of the test mice was observed. The data were processed with SPSS10.0 software, and one-way ANOVA was adopted to verify and compare the distinctiveness of the effect of drug administration. The results showed that AD mice that had been treated with Zn₇MT3 exhibited an improvement in cognitive ability. Compared with the control group, the AD mice in the drug administration group were improved by 20% or more in the residence time is the third quadrant and the frequency at which the mice crossed the original position of the platform, indicating that Zn₇MT3 could effectively prevent the development of the disease of the AD mice under treatment.

### Embodiment 4: Regulation of Zn₇MT3 on Cranial Nerve Cells of AD Mice

After 6 weeks of administration, brain tissues of the AD mice were harvested, fixed in 4% paraformaldehyde, dehydrated, embedded in paraffin, cut into slices with a thickness of 4 microns by a microtome, and then stained with haematoxylin and eosin (HE); and thereafter the cellular morphology in CA1 region of hippocampus was observed under an optical microscope (Leica, Germany). The experimental results were as shown in FIG. 1 (A was a control group, B was an AD mouse model group, and C was a group of AD mouse models intraventricularly administered with Zn₇MT3 in a positioned, sustained-release manner). The cells in CA1 region of the brain tissues of the AD model mice deformed and shrank, no chromatin or ribosome was observed, and the cellular morphology tended to be normal after administration of Zn₇MT3. After the administration of Zn₇MT3, the degeneration of the cranial nerve cells of the mice could be inhibited and the nerve cells were regulated to restore normal functions.

### Embodiment 5: Inhibition of Zn₇MT3 on Aggregation of Amyloid Proteins in the Brain of AD Mice

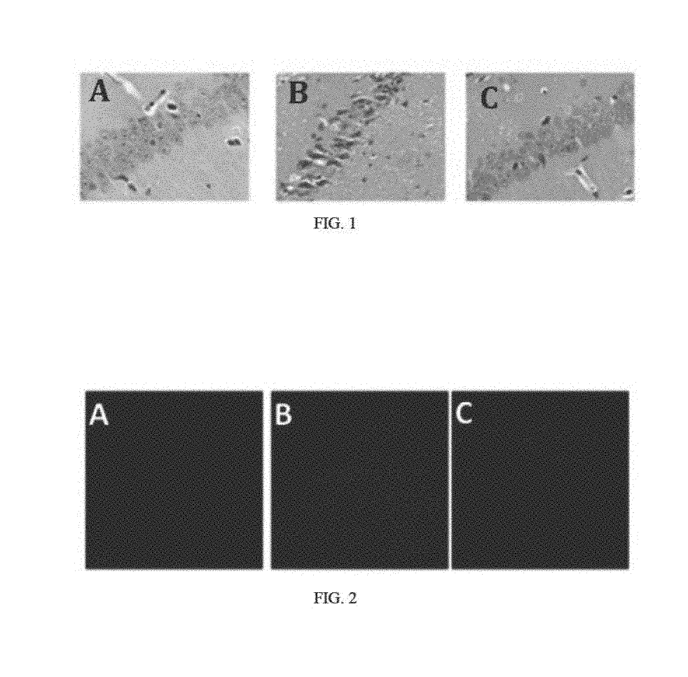

This experiment was thioflavine S staining experiment. The flow of the experiment was as follows: after 6 weeks of administration, brain tissues of the AD mice were harvested, then fixed, embedded in paraffin, sliced, dewaxed in xylene, dehydrated in gradient ethanol and washed with TBS for three times. 0.3% thioflavine S (dissolved in 50% ethanol) was dropped on the tissues, and the tissues were incubated at room temperature for 10 minutes, washed three times with 50% ethanol, washed with TBS, dried, mounted and then observed under a laser confocal microscope. The experimental results were as shown in FIG. 2. Thioflavine S itself has green fluorescence and can specifically bind to mature Abeta amyloid protein, and therefore can be used to label the content and distribution of amyloid protein in brain tissues so as to evaluate the pathological condition of the AD disease. The experimental results were as shown in FIG. 2 (A was a control group, B was an AD mouse model group, and C was a group of AD mouse models intraventricularly administered with Zn₇MT3 in a positioned, sustained-release manner). After administration, Abeta amyloid proteins were significantly reduced, as compared with the model group, which indicated that Zn₇MT3 could remarkably decrease the deposition of Abeta proteins in the brain of the AD mice.

### Embodiment 6: Inhibition of Zn₇MT3 on the Apoptosis of Cranial Nerve Cells of AD Mice

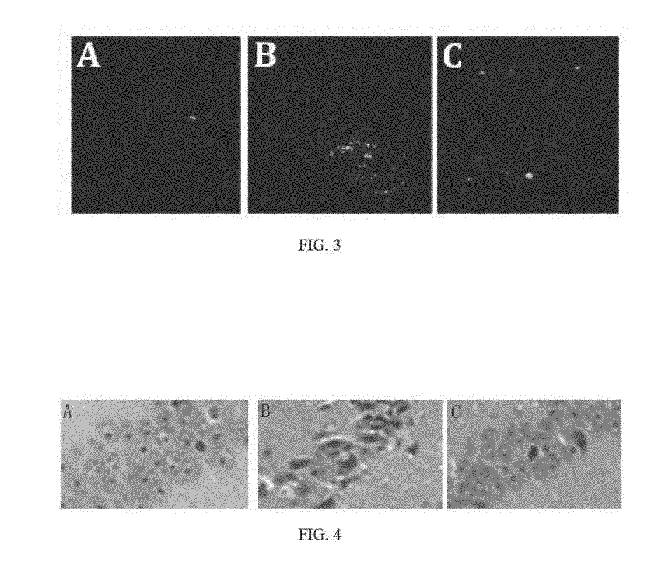

The TUNEL apoptosis detection kit (G3250 kit) was purchased from Promega company. The brain tissues of the mice were harvested 6 week after administration, then fixed, embedded in paraffin, sliced, dewaxed in xylene, dehydrated in gradient ethanol, washed with TBS, incubated with protease K at room temperature for 10 min, sliced, washed with PBS, fixed with formaldehyde, added with an equilibration buffer for preequilibration, washed, then added with an incubation buffer (containing an equilibration buffer, a nucleoside mixture and rTdT enzyme) and incubated at 37° C. for 1 h in the dark, after the reaction was terminated, the resultant product was co-stained with DAPI, dried in the shade, mounted and photographed by a laser microscope. The results were as shown in FIG. 3 (A was a control group, B was an AD mouse model group, and C was a group of AD mouse models intraventricularly administered with Zn₇MT3 in a positioned, sustained-release manner). The results showed that Zn₇MT3 could inhibit the apoptosis of cranial nerve cells of the mice.

### Embodiment 7: Preparation and Characterization of gH625-Zn₇MT3

Unless otherwise specified, all biochemical reagents and kits were purchased from Sigama or Invitrogen company.

(1) Construction of Smt-MT3 Plasmid

Human metallothionein MT3 and fusion tag protein gene Smt3 were purchased from Guangzhou Funeng Gene Company, the gene vector plasmid MBPHT-mCherry-2 was purchased from Invitrogen Company, and the synthesis of the gene primers was completed by Shanghai Generay Biotech Co., Ltd. A PCR product was obtained by amplification using the designed primers P1, P2 (amplifying Smt3 gene) and P3, P4 (amplifying MT3 gene). The amplification product was verified by 1% agarose gel detection and the desired fragment was recovered with gel. Product 1 and product 2 were mixed at the same concentration ratio to serve as a template, and the Smt3-MT3 sequence was obtained by amplification using P1 and P4 primers, with its 5′ end carrying BamH I enzyme digestion site GGATCC and its 3′ end carrying Hind III enzyme digestion site AAGCTT. The designed amplification primers were as follows:

P1:
5′-CGCGGATCCATGGCTAGCATGTCGGACTC-3′
P2:
5′-GGCAGGTCTCAGGGTCCATACCACCAATCTGTTCTC-3′
P3:
5′-GAGAACAGATTGGTGGTATGGACCCTGAGACCTGCC-3′
P4:
5′-CGCAAGCTTTCACTGGCAGCAGCTGCAC-3′

The vector MBPHT-mCherry-2 and the amplified Smt3-MT3 gene sequence were subjected to double enzyme digestion at 37° C. for 6 hours by using BamH I and Hind III endonucleases, respectively, enzyme digestion was verified to be successful by means of 1% agarose gel detection, and the digested fragments were recovered with gel. The digested large and small fragments were mixed at 1:3 and ligated at 16° C. for 8 hours with T4 ligase. The ligation product was transformed into Top10 close competent cells, and positive clones were picked for PCR identification and sequencing to prove successful construction of the fusion protein Smt3-MT3 expression plasmid.

(2) Construction of Smt3-gH625-MT3 Expression Plasmid

Using Smt3-MT3 plasmid as a template and using TOYOBO mutagenesis kit, primers (P5, P6) were designed to insert the gene sequence (H₂N-HGLASTLTRWAHYNALIRAFGGG-CONH₂) of gH625 into the N-terminal of MT3 sequence. The primers were as follows:

P5:
5′-ATTACAACGCACTAATCCGGGCTTTCGGTGGTGGAATGGACCCTGAG
ACCTGCCC-3′
P6:
5′-GTGCCCATCGAGTCAGCGTTGAAGCGAGTCCTAGACCACCAATCTGT
TCTCTGT-3′

The specific experiment operations were as follows:

(a) reverse PCR

1) diluting the primers to 10 pmol/μl, and regulating the concentration of template plasmid DNA to 50 ng/ul;

2) preparing the PCR reaction solution according to the following proportions:

sterilized distilled water 35 μl

10×Buffer for iPCR 5 μl

2 mM dNTPs 5 μl

primer 1 (10 pmol/ul) 1.5 μl;

primer 2 (10 pmol/ul) 1.5 μl;

plasmid DNA (50 ng/ul) 1 μl,

KOD-Plus-1 μl

Total Volume 50 μl

3) performing PCR under the following conditions:

1. 94° C. 2 min

2. 98° C. 10 sec

3. 68° C. 6 min

4. repeating the steps by 2 to 10 cycles:
4° C. Hold

(b) digesting the template plasmid DNA with Dpn I

1) adding 2 ul Dpn I to the PCR reaction solution (total amount of 50 μl) after completion of the PCR reaction, and mixing the solution well gently; and

2) Spinning down, and reacting at 37° C. for 1 hour.

(c) autocyclization of the PCR product

1) melting T4 Polynucleotide Kinase and Ligation high in ice bath, thereafter stirring Ligation high well gently, and spinning down;

2) preparing a reaction solution using a new PCR tube as follows:

Dpn I treated PCR product 2 μl

sterilized distilled water 7 μl

Ligation high 5 μl

T4 Polynucleotide Kinase 1 μl

Total Volume 15 μl

3) stirring well gently and spinning down;

4) reacting at 16° C. for 1 hour; and

5) transforming Escherichia coli with part of the reaction solution, picking monoclones and sequencing.

For the successfully sequenced plasmids, the expressed protein was Smt3-gH625-MT3 fusion protein, the vector was pET22b(+), and the resistance was Kanamycin.

(3) Biological Expression of Smt3-gH625-MT3 Protein and Purification of gH625-MT3

The successfully constructed Smt3-gH625-MT3 plasmid was transformed into host bacterium BL21 (DE3). Single colonies were picked out and placed in 3 ml of LB culture medium, were cultured in a shaker at 37° C. for 8 hours, then inoculated into 50 ml of LB culture medium at a ratio of 1:100 and cultured in a 37° C. shaker for 6 hours, and further inoculated into 2YT culture medium at a ratio of 1.100 (the abovementioned culture mediums all contained 50 μg/ml kanamycin). When the colonies were cultured in a shaker at 37° C. and at 200 rpm to have an OD value of 0.6-0.8, the IPTG inducer was added thereto until the final concentration reached 0.4 mM, and the resultant product was expressed overnight at 16° C.

The purification process of the fusion protein was as follows: the bacteria harvested through centrifugation were suspended (1 ml/g bacteria) by adding thereto Tris buffer solution (20 mM Tris-HCl, 500 mM NaCl, 10% glycerol, 5 mM β-mercaptoethanol, pH=7.5), a small amount of lysozyme, DNA enzyme and 1% PMSF were added thereto, the bacteria were dissolved by stirring and disrupted with an ultrasonic disintegrator, and then centrifuged with a high-speed centrifuge (for 30 min at 12000 rpm), and the supernatant was taken therefrom to flow through a well-balanced Ni-NTA affinity column so that the His-tagged fusion proteins were attached to the column. The impurity proteins were eluted with Tris buffer solution containing 20 mM imidazole until no color change was detected by Coomassie Brilliant Blue. The fusion proteins were eluted with Tris buffer solution containing 200 mM imidazole. The eluted fusion proteins were placed into a dialysis bag for dialysis, the dialysate was Tris buffer solution, thereafter SUMO enzyme was added thereto at a ratio of 1% for enzyme digestion for 6 hours, the resultant product was flowed through the well-balanced Ni-NTA column for three times in total to remove tag proteins and SUMO enzyme, and then subjected to Superdex-G75 gel molecular sieve to further remove the impurity proteins, and the purified gH625-MT3 protein was detected, by 15% SDS-PAGE gel detection, to have a purity of 95% or more.

(4) Preparation of gH625-Zn₇MT3

DTT was added to 2 ml of gH625-MT3 fusion protein solution (5 mg/ml) until the final concentration reached 5 mM, and the mixture was reduced at 4° C. for 2 hours. 6M HCl solution was added thereto to regulate the pH to 1-2, then the mixture was acidified for 1 hour and then was passed through the Superdex G-25 column to remove impurity metal ions, and gH625-MT3 protein was eluted with HCl solution having the pH of 2.0. The effluent was detected by an ultraviolet spectrometer and then collected. Demetallized gH625-MT3 protein solution was loaded into anaerobic glove box after being degassed, DTT was added thereto until the final concentration reached 5 mM, ZnCl₂ having a concentration 20 times the protein concentration was added thereto, 2M Tris base was added thereto dropwise slowly to regulate the pH to 8-9, the product was left overnight at room temperature, and then dialyzed to remove surplus zinc ions, and the fusion metalloprotein gH625-Zn₇MT3 was obtained after protein concentration.

(5) Metal Content Determination of gH625-Zn₇MT3

A small amount of concentrated nitric acid was added to a certain amount of recombinant fusion protein gH625-Zn₇MT3 for nitrification overnight at 65° C., the resultant product was tenfold diluted, and then the content of metal Zn was measured on an inductively coupled plasma optical emission spectrometer (ICP-OES). The results showed that per mole of recombinant gH625-Zn₇MT3 fusion protein contained 7±0.2 moles of Zn.

(6) Removal of Endotoxin from gH625-Zn₇MT3

GH625-Zn₇MT3 was first subjected to an ultrafiltration membrane to entrap endotoxin (LPS) in aggregation state, and then subjected to Polymyxin B affinity column to remove the residual endotoxin.

### Embodiment 8: AD Model Mice and gH625-Zn₇MT3 Drug Therapy

APP/PS1 transgenic mice were purchased from Beijing Zhongke Zesheng Biotechnology Co., Ltd., 5 months old, weighing 24-26 g. Name of the test drug: gH625-Zn₇MT3; solvent: normal saline; and preparation method: preparing the drug into a solution at the desired concentration with normal saline solution just before use. Intraperitoneal administration of mice: the experimental animals were APP/PS1 transgenic mice. The dosage was 2 mg/kg/day, and 6 weeks later, the blood serum and brain tissues were harvested.

### Embodiment 9: Verification of Cognitive Ability of AD Mice Using Morris Water Maze

Installation: a round pool was used, having a diameter of 1 m, a height of 50 cm and water depth of 30 cm, and having a white bottom, and water temperature was maintained at 23±2° C. Four equally spaced points N, E, S and W were marked on the wall of the pool and served as the starting points of the test. The pool was divided into four quadrants, and a platform was placed in the center of the third quadrant (the platform was equidistant from the wall of the pool and the circle center). The platform was submersed in water at 1 cm depth, making the platform invisible. Lots of clues (triangles, squares, circles and rhombuses in different colors were placed in the quadrants) were arranged around the pool and the clues remained unchanged for use by the mice to position the platform.

Place navigation test: the test lasted for 6 days, and training was performed for 4 times at fixed time periods every day. At the beginning of the training, the platform was placed in the first quadrant, and the mice were placed into the pool facing the wall of the pool from any of the four starting points on the wall of the pool. The time it took for the mice to find the platform and the swimming path of the mice were recorded by a free video recording system, and the four times of training referred to the four training sessions started by placing the mice in water from the four different starting points (different quadrants). After the mice found the platform or if the mice could not find the platform within 90 seconds (the latent period was set to 90 seconds), the experimenter directed the mice to the platform, the mice rested on the platform for 10 seconds, and then the next test was started.

Spatial probe test: the platform was removed 24 h after the place navigation test was completed. The mice were then placed into water from the third quadrant, the swimming paths of the mice within 180 s were recorded, the residence time of the mice in the target quadrant (the third quadrant) and the frequency at which the mice crossed the original position of the platform were recorded, and the spatial positioning ability of the test mice was observed. The data were processed with SPSS10.0 software, and one-way ANOVA was adopted to verify and compare the distinctiveness of the effect of drug administration.

The results showed that AD mice that had been treated with gH625-Zn₇MT3 exhibited a remarkable improvement in cognitive ability. Compared with the control group, the AD mice in the drug administration group were significantly improved (from 50% to 78%) in the residence time in the third quadrant and the average frequency at which the mice crossed the original position of the platform, indicating that gH625-Zn₇MT3 could effectively prevent the development of the disease of the AD mice.

### Embodiment 10: Regulation of gH625-Zn₇MT3 on Cranial Nerve Cells of AD Mice

After 6 weeks of administration (gH625-Zn₇MT3 ), brain tissues of the AD mice were harvested, fixed in 4% paraformaldehyde, dehydrated, embedded in paraffin, cut into slices with a thickness of 4 microns by a microtome, and then stained with haematoxylin and eosin (HE); and thereafter the cellular morphology in CA1 region of hippocampus was observed under an optical microscope (Leica, Germany). The experimental results were as shown in FIG. 4 (A was a control group of normal mice, B was an AD mouse model group, and C was an AD mouse model administration group). The cells in CA1 region of the brain tissues of the AD model mice deformed and shrank, no chromatin or ribosome was observed, and the cellular morphology tended to be normal after administration of gH625-Zn₇MT3. After the administration of gH625-Zn₇MT3, the degeneration of the cranial nerve cells of the mice could be inhibited and the nerve cells were regulated to restore normal functions.

### Embodiment 11: Inhibition of gH625-Zn₇MT3 on Aggregation of Amyloid Proteins in the Brain of AD Mice

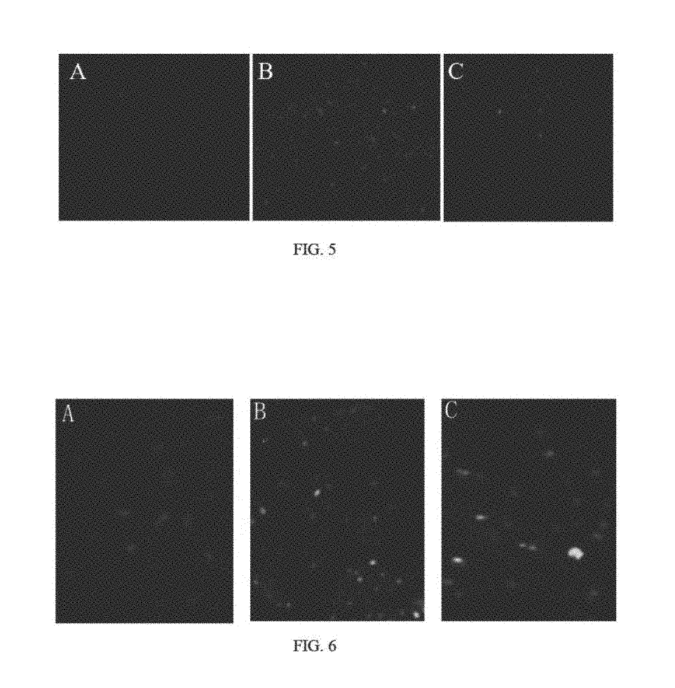

This experiment was thioflavine S staining experiment. The flow of the experiment was as follows: after 6 weeks of administration (gH625-Zn₇MT3), brain tissues of the AD mice were harvested, then fixed, embedded in paraffin, sliced, dewaxed in xylene, dehydrated in gradient ethanol and washed with TBS for three times. 0.3% thioflavine S (dissolved in 50% ethanol) was dropped on the tissues, and the tissues were incubated at room temperature for 10 minutes, washed three times with 50% ethanol, washed with TBS, dried, mounted and then observed under a laser confocal microscope. The experimental results were as shown in FIG. 5. Thioflavine S itself has green fluorescence and can specifically bind to mature Abeta amyloid protein, and therefore can be used to label the content and distribution of amyloid protein in brain tissues so as to evaluate the pathological condition of the AD disease. The experimental results were as shown in FIG. 5 (A was a control group, B was an AD mouse model group, and C was an AD mouse model administration group). After administration, Abeta amyloid proteins were significantly reduced, as compared with the model group, which indicated that gH625-Zn₇MT3 could remarkably decrease the deposition of Abeta proteins in the brain of the AD mice.

### Embodiment 12: Inhibition of gH625-Zn₇MT3 on the Apoptosis of Cranial Nerve Cells of AD Mice

The TUNEL apoptosis detection kit (G3250 kit) was purchased from Promega company. The brain tissues of the mice were harvested 6 week after administration, then fixed, embedded in paraffin, sliced, dewaxed in xylene, dehydrated in gradient ethanol, washed with TBS, incubated with protease K at room temperature for 10 min, sliced, washed with PBS, fixed with formaldehyde, added with an equilibration buffer for preequilibration, washed, then added with an incubation buffer (containing an equilibration buffer, a nucleoside mixture and rTdT enzyme) and incubated at 37° C. for 1 h in the dark, after the reaction was terminated, the resultant product was co-stained with DAPI, dried in the shade, mounted and photographed by a laser microscope. The results were as shown in FIG. 6 (A was a control group, B was an AD mouse model group, and C was an AD mouse model administration group). The results showed that gH625-Zn₇MT3 could inhibit the apoptosis of cranial nerve cells of the mice.

We claim:

1. A use of Zn₇MT3 or a derivative thereof, wherein Zn₇MT3 or a derivative thereof is used for prevention or treatment of Alzheimer's disease or other neurodegenerative diseases, or for development, screening or preparation of a medicament suitable for Alzheimer's disease or other neurodegenerative diseases.

2. The use according to claim 1, wherein Zn₇MT3 or a derivative thereof is used for improving cognitive dysfunction of an AD brain, regulating the cellular morphology of hippocampus in the AD brain, inhibiting the deposition of amyloid proteins in the AD brain or inhibiting the apoptosis of nerve cells in the brain.

3. The use according to claim 1, wherein the dosage form of the medicament comprises at least one of tablets, capsules, granules, suspensions, emulsions, solutions, syrups and injections.

4. The use according to claim 1, wherein the derivative of Zn₇MT3 comprises gH625-Zn₇MT3, or other similar fusion proteins based on metallothionein MT3 or Zn₇MT3 fused with transmembrane small peptide tags.

5. A method for preparing Zn₇MT3, comprising the steps of:
S1, fusion-expressing MT3 with MBP and Smt3 tags; and
S2, subjecting the semi-finished product obtained in S1 to acid denaturation to remove impurity metals, then adding thereto excess zinc ions to renature MT3 protein, and subjecting the resultant product to separation and purification to remove the surplus zinc ions, thereby obtaining Zn₇MT3.

6. The method for preparing Zn₇MT3 according to claim 5, wherein MT3 is solubly expressed in Escherichia coli.

7. A method for preparing gH625-Zn₇MT3, wherein gH625-Zn₇MT3 is made by metal recombination of gH625-MT3 formed by fusion of gH625 and MT3.

8. The method according to claim 7, wherein gH625 comprises a transmembrane sequence is a glycoprotein of herpes simplex virus; the transmembrane sequence contains 23 amino acid residues; MT3 comprises metallothionein III; and metallothionein III contains 68 amino acid residues.

9. The method according to claim 7, wherein a Smt3-MT3 gene expression plasmid containing a fusion tag is constructed by using a vector through the genetic engineering technology; the gene sequence of gH625 is inserted into the Smt3-MT3 gene to form a smt3-gH625-MT3 fusion protein gene; the smt3-gH625-MT3 recombinant fusion protein is solubly expressed in Escherichia coli, and is then subjected to separation and purification to obtain gH625-MT3 recombinant fusion protein; the gH625-MT3 recombinant fusion protein is further caused to bind to zinc ions by chemical recombination, thereby obtaining gH625-Zn₇MT3.

10. The method according to claim 7, wherein the vector is pET22b(+).
